(19) United States
(12) Patent Application Publication
(10) Pub. No.: US 2012/0276056 A1
(43) Pub. Date: Nov. 1, 2012

(54) Method for Use of Biologic Agents Including Live or Dormant Forms of Bacteria and other organisms in Treating Infections, Inflammation and Other Diseases of Distal Small Intestine and Large Intestine

(76) Inventor: Wieslaw Janusz Bochenek, Newtown, PA (US)

(21) Appl. No.: 13/066,844

(22) Filed: Apr. 26, 2011

(51) Int. Cl. A61K 35/74; A61P 31/04; A61P 1/14; A61K 36/062

(52) U.S. Cl. 424/93.4; 424/93.51

(57) ABSTRACT

The present invention relates to method of treating distal gastrointestinal diseases including infections, inflammations and functional motility disorders by oral administration of therapeutic or preventive biologic agent that is released directly to the disease area in the distal ileum, ileacecal junction or colon. The biologic agent may include live or dormant bacteria or other organism, e.g. yeast, or other biologic agent. The delivery vehicle must prevent release of the biologic agent in the upper segments of the gastrointestinal tract where it may lose its potency or decrease in quantity.

### BACKGROUND OF THE INVENTION

This invention pertains to the novel method of administration of probiotic agents in the treatment of distal intestinal diseases.

The use of biologic agents including live organisms and its dormant forms is important as either primary or as adjunctive treatment of diseases of the large intestine (colon) and the distal small intestine. These pathological conditions fall into categories of infections affecting primarily colon, e.g. Clostridium difficile and travelers diarrhea, inflammatory process, e.g. Ulcerative colitis or Crohn's disease, and functional diseases, e.g. Irritable Bowel Syndrome. The subject of this invention is the method of use of biologic agents administered by oral route and destined for release of the active biological constituents in the distal gastrointestinal tract encompassing distal ileum and colon. The release of the active biological agents to the site of disease that is located in the distal gastrointestinal tract is important since the majority of biological agents will be adversely affected in the upper gastrointestinal tract if administered orally. The majority of biologic agents that are not live or dormant organisms will be subject to digestion in the upper gastrointestinal tract that will render them pharmacologically ineffective and thus preventing them to exert their expected therapeutic activity in the distal ileum and or colon. This is the reason why such agents are currently administered using primarily parenteral routes. In case of live organisms, survival of many organisms is limited, if released in the stomach or upper gastrointestinal tract. Not only may they not survive in sufficient quantities the acidic environment of the stomach and high concentrations of bile salts and digestive pancreatic enzymes in the upper small intestine but also will have to compete with indigenous intestinal bacterial flora of the small intestine that will additionally decrease their number or alter their biologic properties. Thus, the quantity and or quality of therapeutic organisms administered orally will significantly decrease before reaching the distal intestinal tract including distal ileum and colon.

One of the best examples of therapeutic potential of probiotic use is Clostridium difficile infection. It is well recognized that ecological bacterial imbalance in the gastrointestinal tract may have significant implications for the health and functioning of the gastrointestinal tract, primarily large bowel, through altering competition in-between the natural benign resident bacterial flora and pathogenic bacteria. Clostridium difficile infection has recently become a significant public health concern. It affects primarily large bowel and accounts for 50-75% of antibiotic associated diarrhea (AAD) and 90-100% of antibiotic associated pseudomembranaceous colitis. The mortality rate of the Clostridium difficile-associated disease (CDAD) is estimated at 6-30% and its increase is attributed to a great extent to the emergence of bacterial strain (NAP-1). It produces 16-23 times more toxin than other strains (8) and responds poorly to metronidazole therapy (9) the standard and less expensive than vancomycin treatment of C. diff. infection. Estimated cost of treatment of hospital acquired CDAD is estimated at $4000 (10, 11). The use of probiotic bacteria, primarily Lactobacillus species and a yeast, Saccharomyces boulardi, has been subject to recent literature reviews and considered effective in lowering the incidence of disease (12) and effective in treating AAD, particularly severe disease (13,14).

Significance of probiotic organisms has been demonstrated not only as a therapeutic agent but also in prevention of AAD and most notably C. diff. infection (15).

Other than infection examples of the benefit of probiotic treatment is inflammatory bowel disease such as Ulcerative colitis (16, 17) and Crohn's disease (18).

Targeted distal intestinal delivery of biological agents will make oral treatment possible for many biologic agents or will ensure their greater therapeutic activity if such oral treatment is currently used. Such therapeutic approach will require special delivery vehicles that will:
a) prevent release of these agents in the upper portions of the gastrointestinal tract prior to reaching distal ileum and/or colon, and
b) in case of live or dormant organisms, will additionally prevent their destruction in the formulation process, so they can be delivered intact to the distal gastrointestinal tract for maximum biologic activity.

### REFERENCES

1.Pepin J, Valiquette L, Cassette B. Mortality attributable to nosocomial Clostridium difficile-associated disease during an epidemic caused by a hypervirulent strain in Quebec. Can Med Assoc J 2005;173:1037-42
2.Archibald L K, Banerjee S N, Jarvis W R. Secular trends in hospital-acquired Clostridium difficile disease in the United States, 1987-2001. J Infect Dis 2004;189:1585-9
3. Sunenshine R H, McDonald L C. Clostridium difficile-associated disease: new challenges from an established pathogen. Cleve Clin J Med 2006;73:187-97
4.Loo V G, Poirier L, Miller M A, et al. A predominantly clonal multi-institutional outbreak of Clostridium difficile-associated diarrhea with high morbidity and mortality. N Engl J Med 2005;353:2442-9
5. Starr J. Clostridium difficile-associated diarrhoea: diagnosis and treatment. BMJ 2005;331:498-501
6. Dubreuil L, Houcke I, Mouton Y, Rossignol J F. In vitro evaluation of activities of nitazoxanide and tizoxanide against anaerobes and aerobic organisms. Antimicrob Agents Chemother 1996;40:2266-70
7. Broekhuysen J, Stockis A, Lins R L, De Graeve J, Rossignol J F. Nitazoxanide: pharmacokinetics and metabolism in man. Int J Clin Pharmacol Ther 2000;38:387-94
8. Warny M, Pepin J, Fang A, et al. Toxin production by an emerging strain of Clostridium difficile associated with outbreaks of severe disease in North America and Europe. Lancet 2005;366:1079-84
9. Spigaglia P, Mastrantonio P. Molecular analysis of the pathogenicity locus and polymorphism in the putative negative regulator of toxin production (TcdC) among Clostridium difficile clinical isolates. J Clin Microbiol 2002;40:3470-5
10. Centers for Disease Control and Prevention. Information for healthcare providers. August 2004. Available from www.cdc.gov/ncidod/dhqp/id_cdiffFAQ_HCP.html. Accessed Dec. 13, 2005
11. Kyne L, Hamel M B, Polavaram R, Kelly C P. Health care costs and mortality associated with nosocomial diarrhea due to Clostridium difficile. Clin Infect Dis 2002;34:346-53
12. Pepin J, Valiquette L, Cossette B. Mortality attributable to nosocomial Clostridium difficile-associated disease during an epidemic caused by a hypervirulent strain in Quebec. Can Med Assoc J 2005;173:1037-42
13. Archibald L K, Banerjee S N, Jarvis W R. Secular trends in hospital-acquired Clostridium difficile disease in the United States, 1987-2001. J Infect Dis 2004;189:1585-9
14. Sunenshine R H, McDonald L C. Clostridium difficile-associated disease: new challenges from an established pathogen. Cleve Clin J Med 2006;73:187-97
15. Loo V G, Poirier L, Miller M A, et al. A predominantly clonal multi-institutional outbreak of Clostridium difficile-associated diarrhea with high morbidity and mortality. N Engl J Med 2005;353:2442-9
16. Starr J. Clostridium difficile-associated diarrhoea: diagnosis and treatment. BMJ 2005;331:498-501
17. Dubreuil L, Houcke I, Mouton Y, Rossignol F. In vitro evaluation of activities of nitazoxanide and tizoxanide against anaerobes and aerobic organisms. Antimicrob Agents Chemother 1996;40:2266-70
18. Broekhuysen J, Stockis A, Lins R L, De Graeve J, Rossignol J F. Nitazoxanide: pharmacokinetics and metabolism in man. Int J Clin Pharmacol Ther 2000;38:387-94
19. Warny M, Pepin J, Fang A, et al. Toxin production by an emerging strain of Clostridium difficile associated with outbreaks of severe disease in North America and Europe. Lancet 2005;366:1079-84
20. Spigaglia P, Mastrantonio P. Molecular analysis of the pathogenicity locus and polymorphism in the putative negative regulator of toxin production (TcdC) among Clostridium difficile clinical isolates. J Clin Microbiol 2002;40:3470-5
21. Centers for Disease Control and Prevention. Information for healthcare providers. August 2004. Available from www.cdc.gov/ncidod/dhqp/id_cdiffFAQ_HCP.html. Accessed Dec. 13, 2005
22. Kyne L, Hamel M B, Polavaram R, Kelly C P. Health care costs and mortality associated with nosocomial diarrhea due to Clostridium difficile. Clin Infect Dis 2002;34:346-53
23. Johnston B C, Supina A L, Ospina M, et al. Probiotics for the prevention of pediatric antibiotic-associated diarrhea. Cochrane Database Syst Rev 2007; 2:CD004827.
   A recent and complete systematic review that reports the efficacy of selected probiotics in the prevention of antibiotic-associated diarrhea and underlines the important role of the dose-response effect.
24. Dendukuri N, Costa V, McGregor M, et al. Probiotic therapy for the prevention and treatment of Clostridium difficile disease: A systematic review. CMAJ 2005; 173:167-170.
25. Czerucka D, Piche T, Rampal P. Review article: Yeast as probiotics—Saccharomyces boulardii. Aliment Pharmacol Ther 2007; 26:767-778.
26. Payne S, Gibson G, Wynne A, et al.: In vitro studies on colonization resistance of the human gut microbiota to Candida albicans and the effects of tetracycline and Lactobacillus plantarum LPK. Curr Issues Intest Microbiol 2003, 4:1-8.
27. Ishikawa H, Akedo I, Umesaki Y, et al.: Randomized controlled trial of the effect of bifidobacteria-fermented milk on ulcerative colitis. J Am Coll Nutr 2003, 22:56-63.
28. Guslandi M, Giollo P, Testoni P A: A pilot trial of Saccharomyces boulardii in ulcerative colitis. Eur J Gastroenterol Hepatol 2003, 15:697-698.
29. Pleain K and Hotz J.: Therapeutic effects of Saccharomyces boulardi on mild residual symptoms in a stable phase of Crohn's disease with special respect

### BRIEF SUMMARY OF INVENTION

The invention pertains to the use of specialized delivery vehicle that will meet the two objectives.
A. The biologic agent in form of live or dormant bacteria or other organism, or other biologic agent have to be kept intact during the formulation process. I can be used free or microencapsulated for additional protection. The most suitable delivery vehicle will be capsule, that does not require “harsh” processing, e.g. compression and temperature changes. Capsule form can be filled with biologic agents including live or dormant organisms using “gentle” processing. Capsules of various sizes can be used to accommodate desired quantities of such agents.
B. The delivery vehicle is such so that it is protected from desintegration in the stomach and the upper small intestine to preserve the biologic agent including live or dormant organisms Preferable release of such biological agent is distal small intestine, or specifically only ileum and/or colon.

### DETAILED DESCRIPTION OF THE INVENTION

Only a few capsules currently available will meet the criteria required by the current invention. Most common gelatin capsule does not meet the required above criteria, since it will disintegrate in the upper gastrointestinal tract and is not suitable for coating to delay capsule disintegration An example of a suitable capsule is a starch capsules that can be manufactured in various sizes, can easily be enteric coated, unlike capsules made of some other materials (Vilivalam V D, Illum I I, Iqbal K: Starch capsules: an alternative system for oral drug delivery. Pharm Sci Technolo Today. 2000 February;3(2):64-69.) This type of capsule is the subject of U.S. Pat. No. 6,228,396 that describes coating of starch capsules which allowed for successful delivery of a pharmacologic agent to the distal ileum and colon. Enteric coating is essential to ensure that the capsules will not disintegrate early but will do so only after reaching the distal ileum and colon. Such enteric coating may consist of a proper combination of Eudragit L100 that dissolves at pH>6 and Eudragit S100 that dissolves at pH>7. In a scintigraphy study, Eudragit L100/Eudragit S100 coated starch capsules were shown to reliably open in the distal intestinal tract (Vinod D. Vilivalam, Lisbeth Ilium and Khurshid lqbal: Starch capsules: an alternative system for oral drug delivery. Pharmaceutical Science & Technology Today Volume 3, Issue 2, 1 Feb. 2000, Pages 64-69). The study demonstrated that approximately 90% of orally administered capsules released their contents in the terminal ileum and colon which includes close to 70% capsules that released their content only in colon. Such pattern of release of biological agents including live or dormant form organisms will ensure that they will be delivered intact to the disease affected area of the distal ileum and/or colon and that the quantity of such agents per delivery unit can be adjusted by the use of capsules of different size.

The present invention therefore discloses method of treatment or prevention of diseases of the distal gastrointestinal tract using biologic agents including live organisms or their dormant forms delivered intact to the affected areas. Such treatment can pertain to gastrointestinal sections of the distal ileum, ileo-cecal junction and colon, and the treatment agent may be administered orally.

1. A method of treatment of distal gastrointestinal diseases such as bacterial infection, e.g. Clostridium difficile or pathogenic Escherichia coli, parasitic infections, e.g. amoebiasis, inflammation, e.g. Ulcerative colitis or Crohn's disease, or functional disease, e.g. Irritable bowel syndrome by oral administration of biologic agent to be released in the distal gastrointestinal tract.

2. The therapeutic or preventive biologic agent in claim 1 may be live or dormant bacteria or other organism, e.g. yeast, or a biologic agent.

3. The biologic agent in claims 1 and 2 is to be delivered to the distal gastrointestinal tract using a delivery vehicle that protects it from premature discharge before reaching the distal ileum and/or colon.

4. The distal gastrointestinal delivery of a biologic agent in claims 1 and 2 is aimed at providing the biologic agents in possibly intact form and in high quantities to maximize therapeutic and/or prophylactic effect.

5. The delivery vehicle can be constructed to have desired characteristics for the preferential release to the gastrointestinal areas, such as the distal ileum, ileo-cecal junction, or colon. This may be based on time needed for the release of the agent, or pH, or some other physicochemical properties of the environment where the agent is to be released.

6. The delivery vehicle may be in a form of capsule, tablet, caplet, or other suitable delivery form that releases the agent passively r actively. Such delivery form can be administered orally or via gastrointestinal tube or through the upper gastrointestinal stoma.

7. The formulation process of the delivery vehicle has to be gentle enough not to damage the biologic agent by mechanical, thermal or any other means.

8. Treatment agents useful with the present invention include biologics that are subject to digestion and thus inactivation or attenuation or loss of therapeutic potency when released in the stomach and/or the upper portions of the small intestine.

9. The delivery method is such that the biologic agent including live organisms can be safely delivered to the affected areas with minimal loss of their quantities or numbers and biologic properties.

10. The treatment agent may exert its therapeutic activity locally in the area of its release such as the distal ileum, ileo-cecal junction or colon, and the therapeutic activity of the agent may be as either intact or in a metabolized form, or both.

11. The therapeutic and/or prophylactic activity of the treatment agent may be related to its systemic therapeutic activity after its absorption and or transformation, or at other times after administration, and the activity of the agent may be as either intact or in a metabolized or a transformed form, or in any combination of the forms.
